(19) United States
(12) Patent Application Publication
(10) Pub. No.: US 2025/0176935 A1
(43) Pub. Date: Jun. 5, 2025

(54) GUIDANCE ASSISTANCE DEVICE FOR ACQUIRING AN ULTRASOUND IMAGE AND ASSOCIATED METHOD

(71) Applicant: DESKI, BORDEAUX (FR)

(72) Inventors: Olivier MOAL, BORDEAUX (FR); Bertrand MOAL, BORDEAUX (FR); Emilie ROGER, LE BOUSCAT (FR); David AUDRAIN, FLOIRAC (FR); Pierre RAGOIS, BORDEAUX (FR)

(21) Appl. No.: 18/862,073

(22) PCT Filed: Jun. 2, 2023
(86) PCT No.: PCT/EP2023/064791

§ 371 Date: Oct. 31, 2024

(30) Foreign Application Priority Data
Jun. 2, 2022 (FR) 2205346

(51) Int. Cl. A61B 8/08; A61B 8/00

(52) U.S. Cl. CPC A61B 8/085; A61B 8/4245; A61B 8/463; A61B 8/5223

(57) ABSTRACT

A guidance assistance device for acquiring ultrasound images, configured to receive in real time ultrasound images acquired by an ultrasound probe connected to the device, and including a first calculator, associated with a first memory, to implement a classifier configured to classify in real time the ultrasound images received and associate with each image a CLASS to generate a quality indicator according to the associated class; a display to display, in real time, the last quality indicator generated; a second calculator, associated with a second memory, and configured to perform the following procedure: determining a relative position of the ultrasound probe relative to a predefined probe target position; the relative position being determined from at least one of the received ultrasound images; and generating and displaying on the display in real-time a position indicator according to the relative position determined.

### FIELD OF THE INVENTION

The present invention relates to a device and a method making it possible to assist the user in acquiring ultrasound images.

### PRIOR ART

Ultrasound imaging, also known as echographic imaging, is a medical imaging technique that uses high-frequency waves to visualize a two-dimensional structure within the body of a living subject. Since ultrasound images are taken in real time, these ultrasound images show the movement of the internal organs in the body as well as the movements of the heart during these beats.

To acquire such images, an ultrasound probe is placed directly on the skin of the subject. A thin layer of gel can be applied to the skin to allow the ultrasound waves to pass through the skin from the probe to the inside of the body of the subject. Ultrasound images are produced by measuring the reflection of ultrasound waves on the organs of the subject. The amplitude of the reflected waves measured, and the flight time of this wave, provide the information necessary to reconstruct the ultrasound image.

During an ultrasound examination, the operator must know where to place the probe and how to orientate it to obtain the desired image. Generally, the desired images of an ultrasound are images for taking certain measurements.

In order to increase ultrasound examination capacity, it has been thought of relieving doctors (general practitioners, cardiologists, radiologists, etc.) or healthcare professionals from taking images. For example, images of views can be taken by an operator who will record the images and then transmit them to the doctor, who will then only have to interpret them. Such an organization makes it possible to increase the number of ultrasound examinations that can be analyzed by a doctor. However, it must be ensured that the ultrasound images are of sufficient quality for interpretation by a doctor. The operator may have ultrasound knowledge, but when he does not perform the image analysis himself, there is a need for a device that assists in taking such ultrasound images.

This acquisition can be difficult for an unfamiliar operator.

Methods implemented by software to guide the operator in taking ultrasound images are known. These methods include probe position detection via position sensors and accelerometers. The software allows an on-screen directional arrow to indicate to the user where to move their probe to improve the quality of the acquired ultrasound image.

One disadvantage of these methods is that it remains difficult for the operator to assess the path to be made to obtain a satisfactory ultrasound image. Another disadvantage is that the user can hardly anticipate the path and sequence of gestures to perform before obtaining a satisfactory ultrasound image. Another disadvantage is that the operator has no idea, when an arrow is displayed, if the distance to travel is small or large. The operator must therefore try to find the correct probe position. Another disadvantage is that the gaze of the examiner remains focused on the arrow and thus loses sight of the ultrasound image that he is acquiring.

Also known is EP3868303, which describes a method based on the quality trend of a series of images to provide an indication to the user.

A disadvantage of this method is that it may fail to guide the user to a target position, for example if the user comes across a local extremum. Another drawback is that this method does not generate indications in real time: enough images must have been acquired at different positions to detect a quality trend.

Also known is US20154/327838, which describes the detection of regions of interest in ultrasound images.

One drawback is that this method requires such regions of interest to be present on the acquired ultrasound image, which is not the case if the probe position is too far from a target position.

There is therefore a need for a new device making it easier for the operator to take ultrasound images.

The invention therefore aims to provide a device and an associated method allowing operators to acquire ultrasound image sequences of sufficient quality more easily and more quickly.

### SUMMARY OF THE INVENTION

According to one aspect, the invention relates to a guidance assistance device for acquiring ultrasound images, in particular ultrasound images of the heart, configured to receive in real time ultrasound images (in particular of the heart) acquired by an ultrasound probe connected to said device, and comprising:
a first calculator, associated with a first memory, to implement a classifier configured to classify in real time the ultrasound images received and associate with each image a class to generate a quality indicator according to said associated class;
a display to display, in real time, the last quality indicator generated;
a second calculator, associated with a second memory, and configured to perform the following steps:
   determining a relative position of the ultrasonic probe relative to a pre-defined probe target position, said relative position being determined from at least one of the received ultrasound images; and
   generating and displaying on the real-time display of a position indicator according to the relative position determined.

According to the display is configured to display the received ultrasound images in real time.

One advantage is to allow the user to visualize the deviation between the position of their probe and a position allowing them to acquire a desired ultrasound image. In addition, such a position indicator, generated according to the relative position, allows the user to both estimate a distance to travel as well as the direction to take with a single indicator. This allows the user to acquire the desired ultrasound image more quickly and easily.

According to one embodiment, the first and the second are the same memory. Thus, in the present application, a memory is referred to indiscriminately as a physical device for storing data or an available memory space of a physical device.

According to one embodiment, the first calculator and/or the second calculator is/are locally included in the device of the invention. According to another mode, the first calculator and/or the second calculator is included in a remote equipment such as a data server. In the latter case, the device of the invention is understood as a system comprising several pieces of equipment. In one embodiment, the first and second calculators are confounded or implemented in the same computing device.

In one embodiment, the classifier is configured to classify an image among a plurality of classes, each representing a particular view of the organ, and at least one class representing an insufficient quality view.

The advantage is to make it possible to detect both a particular view and images that would not be usable by the doctor to make a diagnosis.

In one embodiment, the at least one class representing an insufficient quality view comprises at least one ultrasound image class comprising enough information (e.g. anatomically visible zones) to enable the determination of the relative position of the probe with respect to a target position or a reference position.

In an embodiment, the classifier is implemented by means of a learner function configured from supervised machine learning.

In one embodiment, the step of determining the relative position of the ultrasonic probe relative to a predefined target position of the probe from the received ultrasound images is implemented by means of a learner function configured from supervised machine learning.

In one embodiment, said determination step is implemented by means of a neural network trained by a series of ultrasound images, in particular of the heart, labeled. The advantage of such a classifier is that it allows reliable classification of ultrasound images from training from labeled images.

According to one example, the neural network is a convolutive neural network, known as CNN or Convet.

According to one embodiment, the configuration of the neural network CNN may comprise:
Convolutions or neural network layers comprising a plurality of multiplications of matrices comprising weighting coefficients obtained from a learning method,
non-linear operations.

According to one embodiment, the configuration of the neuron network CNN comprises as input(s) images acquired or received or stored in a memory.

The neural network CNN may comprise convolutions in its first layers, then fully connected layers of neurons at the end of the model. In the latter case, they are neurons connected to all neurons in the previous layer and connected to all neurons in the next layer.

The convolution layers may comprise a scan of an input matrix producing a series of matrix calculations. The other layers of the neural network typically comprise matrix calculations on the size of the input matrix.

According to one example, each convolution comprises a matrix product between an input matrix and a weight matrix and the consideration of an additional bias.

The application of successive layer processing within the neural network CNN comprises the application of a series of matrix multiplications which are followed by a non-linear function to produce an output of said layer. The succession of these operations defines the depth of the neural network.

According to one example embodiment, the neural network is a multilayer perceptron, known as MLP. According to one example, the neural network may be a network equivalent to MLP.

In one embodiment, said labels comprise relative spatial coordinates of the position of the probe with respect to a target position and/or relative orientation information of the probe with respect to target orientations in an orthonormal coordinate system.

In one embodiment, the predefined probe target position is pre-recorded, preferably in the second memory. In one embodiment, the target position is associated with a predefined image class.

In one embodiment, the displayed position indicator comprises a first visual element and a target mark; the position of said first element displayed on the display with respect to the target mark being a function of the relative position of the determined probe.

In one embodiment, the relative position determined comprises at least one relative orientation information of the probe with respect to a target orientation with respect to a predetermined axis of an orthonormal coordinate system; and wherein the orientation of said first visual element with respect to the orientation of the target mark is a function of said relative orientation information.

One advantage is to allow the user to visualize on a single visual element the probe placement deviation and a probe orientation deviation to acquire a desired ultrasound image. This allows the user to acquire the desired ultrasound image more quickly and easily.

In one embodiment, the determined relative position reaches a predefined threshold value, the first visual element is confounded with the target mark.

In one embodiment, when the acquired image is classified into the predefined image class, the first visual element is confounded with the target mark. According to one embodiment,

In one embodiment, the target mark is arranged on the ultrasound image displayed in real time.

One advantage is to allow the user to view both the ultrasound image acquired and the position indicator in the same location.

In one embodiment, the target mark comprises the contours of the displayed ultrasound image; the dimensions of the first visual element being similar to the dimensions of the contours of the displayed ultrasound image.

In one embodiment, the target mark comprises displaying a target and wherein the first visual element position relative to the center of the target is according to the determined relative position.

In one embodiment, the position indicator further comprises a 3-dimensional view of an organ; the target mark comprising a first target plane passing through said displayed organ and the first visual element comprising a second plane passing through said displayed organ.

According to another aspect, the invention relates to a system comprising a device according to the invention and an ultrasonic probe. The ultrasonic probe is preferably capable of being connected to said device in order to transmit the ultrasound images acquired by the probe to the device.

According to another aspect, the invention relates to a guidance assistance method for acquiring ultrasound images, in particular ultrasound images of the heart. Said method is implemented by computer.

Said method comprises the following steps:
continuously receiving ultrasound images (11) from an ultrasound probe;
real-time classification of received ultrasound images, and association to each image in a class;
generating and displaying (300) a quality indicator according to said class associated with the classified ultrasound image;
detecting a relative position of the ultrasonic probe relative to a predefined probe target position from the received ultrasound images; and
generating and displaying on the real-time display of a position indicator according to the relative position determined.

In one embodiment, the method comprises displaying received ultrasound images continuously or in real time on the display.

In one embodiment, the position indicator is displayed superimposed on the displayed ultrasound image. In one embodiment, the position indicator is displayed superimposed on a portion of the human body displayed on the display.

According to another aspect, the invention relates to a device comprising software and hardware means for executing the method according to the invention. The hardware means may comprise a display, a receiver, an ultrasound probe, one or more processors or calculators associated with memories, one or more memories to store the ultrasound image sequences, and/or a transmitter.

According to another aspect, the invention relates to a computer program product comprising instructions that lead the device according to the invention to execute the steps of the method according to the invention.

According to an aspect, the invention relates to a computer readable support, on which the computer program according to the invention is recorded.

### BRIEF DESCRIPTION OF THE FIGURES

Other characteristics and advantages of the invention will become clearer on reading the following detailed description, in reference to the appended figures, that illustrate:

FIG. 1: a schematic view of the image displayed by the display of the device according to a first embodiment of the invention.

FIG. 2: a schematic view of a device according to an embodiment of the invention.

FIG. 3A: a schematic view of the image displayed by the display of the device according to a second embodiment of the invention.

FIG. 3B: a schematic view of the image displayed by the display of the device according to a third embodiment of the invention.

FIG. 3C: a schematic view of the image displayed by the device according to a fourth embodiment of the invention.

FIG. 3D: a schematic view of the image displayed by the device according to a fifth embodiment of the invention.

FIG. 4: a flowchart representing the different steps of a method according to one embodiment of the invention.

### DETAILED DESCRIPTION

The invention relates to a method 1000 of assisting in generating a sequence of ultrasound images.

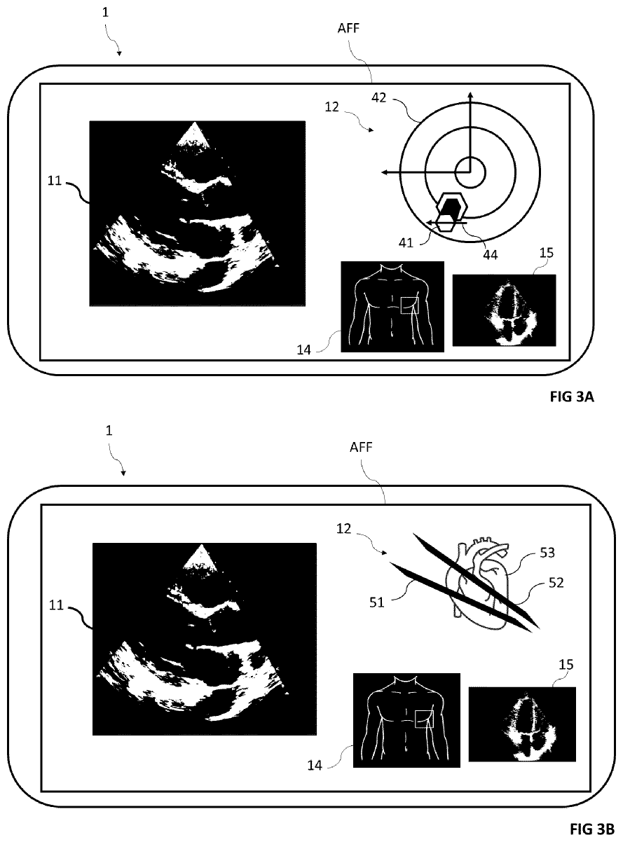

The invention also relates to an associated device. An example of such device 1 is shown in FIG. 2.

Device 1 may comprise a tablet, a smartphone, a computer, or any other device comprising at least one display and a processor associated with a memory. According to another aspect, the invention also relates to a system 2 comprising a device 1 according to the invention and an ultrasonic probe SECH connected to said device 1.

In one embodiment, the device comprises software and hardware means for implementing the method according to the invention described below. Said device preferably comprises at least one receptor REC, a classifier, and a display.

### Reception

The method comprises a step of receiving 100 ultrasound images 11 in real time. Said ultrasound images are received by a receptor REC of the device. In one embodiment, the receiver REC may comprise a buffer memory wherein images received 11 by the receiver REC are temporarily stored prior to transmission.

The received ultrasound images 11 are displayed in real time, preferably on a display AFF of the device. The received ultrasound images can therefore be transmitted to a display for display in real time.

The device includes a display AFF. The display AFF may comprise a screen such as a monitor, a touchscreen tablet or a smartphone.

The display AFF is connected to the receiver REC and/or the classifier CLASS and/or the processor. The display AFF is configured to display in real time the ultrasound images received by the receiver REC. The display AFF advantageously allows the operator to have real-time feedback on the ultrasound images that they are acquiring. The display AFF is configured to show additional indicators or data that will be described later in this description. The display AFF displays an image 1 comprising an ultrasound image received 11 by the receiver REC. Preferably, the ultrasound image displayed is the last ultrasound image received by the receiver REC. The display AFF is thus configured to display in real time the images captured by the ultrasonic probe SECH connected to the device according to the invention.

### Classification

The method comprises a step of classifying 200 of the ultrasound images 11 received by the receptor REC in real time. Classification is performed by a classifier CLASS of the device.

The classification of an ultrasound image comprises associating said ultrasound image with a class 14, preferably a class among a predefined class group.

In one embodiment, the classification further comprises generating 300 a quality indicator 13 according to said class associated with the ultrasound image. The quality indicator may be representative of a class or predefined set of classes.

In one embodiment, the step of classifying further comprises generating a confidence value. The confidence value may represent the confidence rate of the classification performed. The confidence value is generated by the classifier CLASS.

The classification step is preferably performed by a classifier CLASS of the device. Classifier means an algorithmic function executed by a processor or a calculator associated with a memory.

The classifier CLASS is configured to receive images received by the receiver REC.

The classifier is configured to classify in real time at least part of the images received by the receiver REC. The classifier is configured to associate a class with an ultrasound image. The classifier generates a quality indicator 13 according to the associated class. The quality indicator 13 is preferably associated with said classified ultrasound image.

Preferably, the image processing means device for processing the images received before providing them to the classifier CLASS. These processing means may include image filters or contrast functions.

Preferably, the classifier CLASS is configured to classify an ultrasound image among a plurality of classes comprising, on the one hand, several or at least one class each representing a particular view of the organ and on the other hand, at least one class representing an insufficient quality view. In one embodiment, the at least one class representing an insufficient quality view comprises a class representing a view the quality of which allows determination of the relative position of the probe as described below.

Thus, when an image is classified in a class representing a particular view of the organ, this image is considered of sufficient quality to advantageously enable the clinician to take predefined measurements from these images.

In one embodiment, each class representing a particular view of the organ represents a particular view of the heart from:
The view of a large axis parasternal section
The view of a small axis parasternal section (aortic, mitral, pillar)
The view of an apex section (2, 3, 4 and 5 cavities)
The view of a subcostal section (4 cavities, inferior vena cava)
The view of a suprasternal section
The view of a straight paraphernal section

Each of these views is well known to cardiologists and is used to visualize different parts of the heart, calculate or measure specific data, and identify certain pathologies.

Each of these views can be characterized by the presence of one or more specific parts of the heart. For example, the view of a small axis parasternal section is characterized by the presence of the left ventricle and right ventricle section on the image. From this view, the cardiologist can calculate the shortening fraction and calculate lung pressures.

In one embodiment, the class representing an inadequate quality view represents ultrasound views of the heart not belonging to one of the specific views of the other classes or specific ultrasound views listed above, but the image quality of which does not allow the necessary measurements related to this specific view to be made.

The invention is particularly advantageous in the context of acquiring ultrasound images of the heart since the heart requires the taking of specific images well known to practitioners as explained previously.

However, the invention may also find advantages in the ultrasound acquisition of other organs requiring assistance in guiding the user to obtain a characteristic view of said organ. For example, the invention also finds its interest in acquiring ultrasound images of the lungs, liver, uterus or in the case of an abdominal ultrasound or obstetric ultrasound. In one embodiment, the “organ” comprises a human or animal fetus or an organ of such a fetus.

In an embodiment, the classifier CLASS is implemented by means of a learning function trained from supervised and/or automatic learning. The learning function preferably comprises a neural network. The learning function is preferably trained by a series of ultrasound images of a labeled organ.

In an example, the learner function was configured from a learning comprising submitting to the classifier a plurality of ultrasound images each associated with a label. In one embodiment, the classifier was trained with ultrasound images of a specific view of an organ of sufficient quality, each image being associated with a label representing said specific view. The classifier was also trained with ultrasound images of organ views other than those cited above or not of sufficient quality allowing the physician to make measurements from these images, each of these images being associated with a quality label representing a view of insufficient quality. The labels may also comprise information characteristic of taking an image of an organ comprising a viewing angle and/or a characteristic size, an image quality, the presence of a specific portion of an image of the organ, the presence of a contour with a measurable anatomical shape. Preferably, the labels comprise the name of the particular view of the organ or a name associated with a poor quality view.

In another embodiment, the ultrasound images used for learning may comprise ultrasound images representing particular views of another organ.

The classifier CLASS can be run in a processor itself associated with a memory. The classifier CLASS may be stored in a computer-readable medium such as a memory associated with said processor.

In one embodiment, the step of classifying an ultrasound image comprises generating a correspondence score for each class. The correspondence score may comprise a probability that the ultrasound image belongs to each class. The method then includes selecting the class with the highest score. Preferably, the confidence value is generated from said correspondence score of the class associated with said ultrasound image.

In an embodiment, the classifier CLASS classifies 100% of the images received in real time.

In an alternative embodiment, the classifier CLASS is configured to classify only one rate of received images. The classifier is then configured to classify a portion of the received ultrasound images in real time. This embodiment is particularly advantageous when the classification speed is less than the image acquisition speed (in images per unit of time).

In one embodiment, the classification of an ultrasound image comprises the classification of the last received ultrasound image. When said image has been classified, the method again takes the last received ultrasound image. As a result, some ultrasound images may not be classified. However, this execution mode advantageously makes it possible to classify ultrasound images in real time, regardless of the speed of the classifier or the frequency of reception of the ultrasound images.

### Quality Indicator

The method according to the invention comprises generating and displaying in real time on the display AFF of the quality indicator 13 generated. Preferably, the quality indicator 13 displayed corresponds to the last quality indicator 13 generated.

In one embodiment, this quality indicator 13 is generated by the classifier CLASS. Such a classifier is configured to, when receiving an ultrasound image of the organ, classify said image into one of the classes mentioned above. The method then comprises generating a quality indicator representative of the classification of this image. Said quality indicator may be associated with said classified ultrasound image.

Alternatively, the quality indicator 13 can be generated by a remote processor connected to the display AFF and receiving information from the classifier.

The display of the quality indicator 13 may comprise the display of a colored indicator the color of which depends on the class associated with the classified ultrasound image. The color of the indicator may be representative of a single class. Preferably, the color of the colored indicator may take two distinct colors, a first color representative of a class group including the classes representing a particular view of the organ such as those mentioned above, and a second color representative of a class representing an insufficient quality view. Thus, the operator can advantageously see more quickly whether the ultrasound image they are taking is of sufficient quality or not.

In one embodiment, the classifier is also configured to, when it receives an ultrasound image of the organ, generate a confidence value 21. The confidence value 21 may be representative of a level of certainty that the image has been classified correctly. In one embodiment, the quality indicator may comprise said confidence value. The confidence indicator 21 is preferably displayed by the display AFF. The quality indicator 13 associated with the classified image may include said confidence indicator.

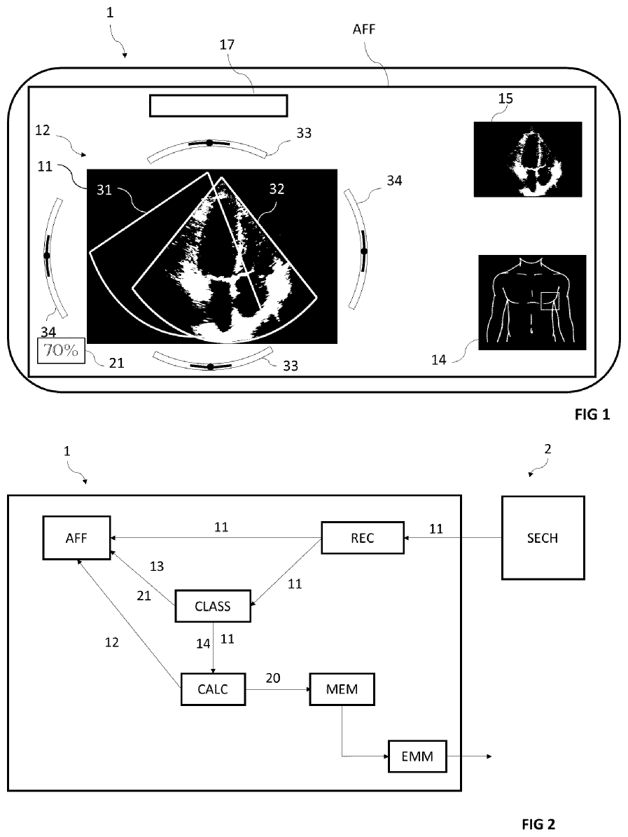

In a first example, the displayed quality indicator 13 comprises a colored frame, which preferably extends around the displayed ultrasound image. The operator can then advantageously view whether the image they are taking is the one expected without having to look away from the image. The confidence indicator 21 may be displayed as a numerical value as shown in FIG. 1.

In a second example, the quality indicator 13 comprises a colored indicator. The color of the colored indicator is representative of the classification of the last ultrasound image. At least one of the dimensions of said colored indicator is according to the confidence value. In the example shown in FIG. 1, the quality indicator is a bar 17 the length of which varies according to the confidence value and the color of which depends on the class associated with the last ultrasound image.

Such an indicator advantageously allows the operator to view whether the image they are taking is of sufficient quality and also allows them to view without diverting their gaze if the indicator is degraded or improved according to the movement of the ultrasound probe SECH. The purpose of such an indicator is to allow the operator, when moving the probe slightly, to visualize whether this movement increases or decreases the confidence value, and therefore increases or decreases the likelihood of obtaining a validated sequence of images described below.

### Sequence

The method may comprise automatically recording 400 a sequence 20 of images in a memory MEM. This sequence 20 is automatically recorded when a sequence of a predefined number of received or classified images comprises a rate of images associated with the same class 14 above a predefined rate. Preferably, said class is a class representing a particular view of the organ.

“Image sequence” means a series of ultrasound images that follow in chronological order of their acquisition. The term “video sequence” is therefore used to refer to such a sequence of images.

Thus, when a sufficiently long video comprises, for example, a majority of ultrasound images classified in the same class representing a particular view of the organ, the video sequence 20 is automatically recorded in a memory. This automatic recording advantageously automatically generates videos of a particular view of the organ that can be analyzed by a cardiologist without operator validation.

The predefined number of received or classified images can be understood as a predefined minimum duration, provided that the image acquisition frequency of the ultrasonic probe SECH is constant. The advantage of this threshold is to ensure that the video sequence 20 is long enough to be analyzed by a doctor. In another embodiment, the predefined number of images may be replaced by a number of received or classified images. For cardiology, the predefined number can be set to correspond to a predefined number of cardiac cycles of the heart of the subject.

The rate of images associated with the same class in said video sequence greater than a predefined rate advantageously allows automatic recording despite a negligible number of images not having sufficient quality in said sequence. Indeed, this negligible number of images in a video sequence can come from a classification error, or noise linked to a particular image. This tolerance advantageously consists of a good compromise between the ease of automatic generation of a sequence and the quality of said sequence.

In one embodiment, the predefined rate is at least greater than 50%. In another embodiment, the predefined rate can be set by the operator, for example by a user interface of the device.

In one embodiment, the automatic recording step comprises buffering the continuously received ultrasound images as well as the generated quality indexes associated with said images. In this way, when a video sequence fulfilling the above criteria is detected, the images belonging to that sequence can be transferred from the buffer memory to another memory, and/or grouped in the same file to generate a video sequence.

### Position Determination

According to one embodiment, the method according to the invention comprises displaying at least one position indicator 12. The position indicator 12 is intended to assist the user in guiding the probe to obtain ultrasound images that can be classified into a predetermined class.

The generating of a position indicator 12 comprises the detecting in real-time of the relative position of the ultrasonic probe with respect to a pre-recorded target position.

Said target position is preferably associated with a class of ultrasound images or a quality indicator such as described above.

In a first embodiment, the relative position of the probe is obtained by means of detecting the probe, such as an optical detection device and/or acceleration sensors embedded in the probe.

In a second preferred embodiment, the relative position of the probe is determined in real time from the acquired images 11 received from the ultrasonic probe SECH. In one embodiment, a calculator is configured to perform a learning function such as a deep learning program. The learner function may be similar to the one described above.

The learning function is trained from labeled ultrasound images, each training ultrasound image being associated with a relative position of the probe wherein said image was acquired with respect to the target position. Said function is configured to receive ultrasound images as input and to generate as output a relative position of the probe with respect to said target position, preferably by a regression operation.

Preferably, the function is configured to receive ultrasound images and to generate, for each ultrasound image received, a relative position of the probe with respect to a target position.

Preferentially, said learning function implements a classifier configured to classify the received ultrasound images in real time and associate with each image a relative position with respect to a predefined target position and to generate a position indicator as a function of said associated relative position.

In one embodiment, the labeled ultrasound images for training the learning function were generated by acquiring ultrasound images while calculating the real-time position of the ultrasound probe, for example with an optical camera.

Said relative position may comprise two spatial coordinates of X positions, where Y represents the relative position of the probe head relative to the target position on the surface of the body of the subject.

Said relative position may further comprise relative orientation information T1, T2, T3 of the probe with respect to target orientations. Preferably, the relative position comprises 3 relative orientation information T1, T2, T3. Each relative information represents an angular deviation between the orientation of the probe relative to the target orientation along an axis of an orthonormal coordinate system.

In one embodiment, the learning function is therefore configured to associate spatial position coordinates and/or 3 probe relative orientation information with each received ultrasound image, each relative orientation information of the probe representing an angular deviation between the orientation of the probe relative to the target orientation along an axis of an orthogonal reference frame. In this embodiment, said function has preferably been previously trained by labeling training ultrasound images where each training ultrasound image was associated with two spatial position coordinates and/or 3 relative orientation information of the probe.

The generation of the position indicator comprises the display in real time of the position indicator according to the relative position determined in real time.

In one embodiment, the first visual element 31 is displayed on the screen and the position of this visual element on the display in relation to a target point of the display depends on the determined relative position.

More precisely, a reference position is determined on the AFF display (preferably displayed) and the spatial coordinates of the first visual indicator with respect to the reference position depend on the spatial position coordinates of the determined relative position. For example, the deviation between the first visual element 31 and the target position depends on the relative position.

This not only gives the user the advantage of knowing in which direction to point their probe, but also gives the user an idea of how far to travel to reach the position allowing them to obtain the desired ultrasound image.

In a first example shown in FIG. 1, the visual element comprises a geometric shape and the target position is visualized by the contours 32 of the ultrasound image. This example is particularly advantageous since, in addition to giving the user a good idea of the relative position, the user can more easily visualize both the relative position indicator and the ultrasound image that they are acquiring.

Preferably, said geometric shape of the visual indicator is similar to that of the contours 32 of the ultrasound image 11 displayed in real time. Thus, the user knows that the probe has reached the target position when the visual element 31 is confounded with the contours 32 of the ultrasound image 11.

In one embodiment, the orientation of the visual element 32 is according to a relative orientation information T1 along a first axis, preferably with respect to a normal axis with respect to the surface of the body of the subject. This advantageously allows the user to view two indications simultaneously on the same visual element 31. In this mode, the geometric shape is not circular. For this purpose, the characteristic pie chart shape of the ultrasound image is particularly advantageous.

In one embodiment, the position indicator comprises one or two other visual elements 33, 34 representing the relative orientation information T2, T3.

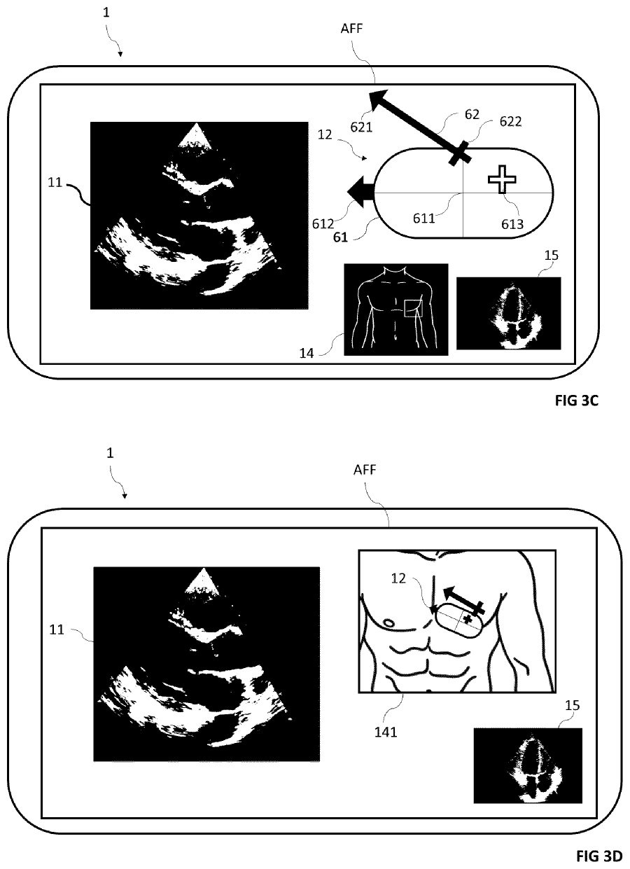

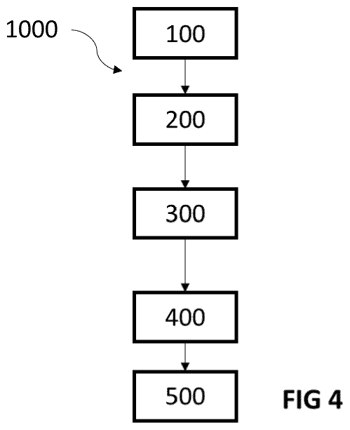

In a second example shown in FIG. 3A, the visual indicator comprises a target the center of which represents the target position and an element the position of which relative to the center depends on the relative position of the determined probe. Said element may comprise a virtual probe 41. The virtual probe may comprise an orientation indicator 44 to allow the user to view the orientation of the virtual probe along an axis normal to the displayed target 42.

Preferably, the top of the virtual probe shifts from its base according to the relative orientation information T2, T3.

In a third example shown in FIG. 3B, the visual indicator comprises a view of a 3-dimensional organ 53 and a target plane 52 passing through said organ and further comprises a moving plane 51 the position of which with respect to the target plane depends on the determined relative position.

In a fourth example shown in FIG. 3C, the visual indicator comprises a target 62 and a marker 61. The target comprises a first visual cue 622 designed to indicate a position and comprises a second visual cue 621 designed to indicate a direction. In the example shown in FIG. 3C, the first visual cue comprises a cross and the second visual cue comprises a direction.

Marker 61 comprises a first visual cue designed to indicate the relative position of the probe with respect to the position of the target according to two translational degrees of freedom. In the illustrated example, the first visual marker 611 is formed by the intersection of two lines and/or is formed by the center of a geometric shape representing the sensor of the ultrasound probe head.

Marker 61 further comprises a second visual cue designed to indicate a direction, such as an arrow 612. This second visual cue is intended to indicate a direction of the probe. In one embodiment, the first visual cue the geometric shape used, if non-circular, may also serve to indicate said direction. The second visual marker and the first visual marker are preferably displayed fixed relative to each other.

The marker further comprises a third visual cue 613 designed to indicate a probe orientation.

In the embodiment shown in FIG. 3C, the target 62 is displayed fixed on the display.

The east marker 61 is displayed movably by the display. The coordinates of the first visual marker 611 in relation to the first visual marker 622 of target 62 depend on the relative position of the probe in relation to the predefined position of the probe to obtain an image of good quality.

Advantageously, this second visual mark indicates to the user the orientation to be given to the probe along an axis perpendicular to the plane of the probe head sensor to obtain an image considered of sufficient quality.

Finally, the position of the marker's third visual indicator 613 is displayed movably relative to the first visual indicator 611 and the second visual indicator 612 of marker 61. The third visual marker 613 is designed to display a position on the display. The position of the third visual indicator 613 of the marker relative to the position of the first visual indicator 611 of the marker 61 is a function of a deviation between the inclination of the probe relative to the skin surface in two degrees of freedom.

In short, the probe on the body can be moved with 2 degrees of translational freedom and 3 degrees of rotational freedom. Each target position of the probe, enabling acquisition of an image of satisfactory quality, is therefore predefined by 2 coordinates and 3 orientations.

The relative position of the probe is therefore defined by 2 coordinate values and by 3 orientation values according to the 3 rotational degrees of freedom.

The relative position on the display of the first visual marker 611 of marker 61 in relation to the first visual marker 622 of target 62 is a function of the two translational coordinates of the relative position of the probe.

The angle formed between the direction of the second visual marker 621 of target 62 and the direction of the second visual marker 612 of marker 61 is a function of a first orientation value according to a first rotational degree of freedom of the relative position of the probe, preferably according to an axis parallel to the longitudinal axis of the probe.

Finally, the relative position on the display of the third visual marker 613 of marker 61 with respect to the first visual marker 611 of marker 61 is a function of the value of the two other orientations according to the two other degrees of freedom in rotation, preferably according to the two axes orthogonal to the axis parallel to the longitudinal axis of the probe.

In the example shown in FIG. 3C, the user will move the probe so as to superimpose the positions of the first visual marker 622 of target 62 and the first visual marker 611 of marker 61, rotate the probe clockwise or counterclockwise to align the arrow 621 of the target with the arrow 612 of the marker, and tilt the probe to superimpose the first visual marker 611 of marker 61 with the third visual marker 613 of marker 61.

Preferably, marker 61 represents an oval representing the probe head sensor and third indicator 613 represents the extension of the probe's longitudinal axis.

In one embodiment shown in the various FIGS. 3A, 3B, 3C, the position indicator 12 is displayed alongside the acquired ultrasound image 11.

In another embodiment not shown, position indicator 12 is displayed superimposed on acquired ultrasound image 11.

In another embodiment shown in FIG. 3D, position indicator 12 is superimposed on a representation 141 of a human body. In this embodiment, displaying the ultrasound image on the display is optional.

### Program

According to one embodiment, the method according to the invention comprises selecting a first class from classes representing a particular view of the organ, for example one of the views of the heart mentioned above.

Selecting the first class generates the display on the display AFF of a first ultrasound image 15 of the pre-recorded organ representing such a view. Preferably, the label 17 that may comprise the name of said particular view is also displayed on the display AFF. Selecting the first class also generates the display of a first setpoint image 14. The first setpoint image 14 is preferably pre-recorded. The setpoint image shows a position and/or orientation setpoint of the ultrasound probe SECH on a patient to obtain said view corresponding to the class selected. The operator is thus advantageously guided and assisted in taking such a view. On the same display AFF, they can view an example of the view that they must take, the position and orientation of the probe to achieve this. Finally, the operator can view in real time whether the view acquired is of sufficiently good quality by the quality indicator 13, and can advantageously view the influence of the movements they perform on the quality of the image acquired thanks to the confidence indicator.

Finally, once the operator has found an image of satisfactory quality, they are assisted in real time by the time indicator representative of the time during which they must hold the probe to acquire images of sufficient quality. The operator is also assured, by viewing the time indicator, that a first sequence 20 will be recorded while maintaining their position.

Once the first sequence has been recorded or validated, the method may comprise automatically selecting a second class from the classes representing a particular view of the device. Again, selecting said second class automatically generates the display of a second pre-recorded setpoint image and the display of a second pre-recorded ultrasound image showing an ultrasound view.

According to one embodiment, the method further comprises generating and displaying a progress indicator. The progress indicator may be representative of the number of sequences that have been recorded. For example, the progress indicator is representative of the number of classes representing a particular view of the organ for which an image sequence 20 has been generated and/or recorded automatically according to the method according to the invention.

According to one embodiment, the method comprises displaying the recorded sequence and further comprises a second manual validation by the operator after said sequence is displayed.

### Device

According to one aspect, the device according to the invention comprises software and hardware means for implementing the method such as described above.

An embodiment of the device according to the invention is now described in reference to FIG. 2.

The device includes a receptor REC. The receiver REC is intended to be connected to an ultrasonic probe SECH in such a way as to receive continuously and in real time ultrasound images 11 acquired by said ultrasonic probe SECH.

The receiver REC can be connected to the ultrasonic probe SECH by a wired or wireless link, for example by a Bluetooth connection or a WI-FI connection or any other data exchange protocol known to those skilled in the art.

The receiver REC may comprise or be associated with one or more memories to temporarily store the images received. The receiver REC is directly or indirectly connected to the display AFF to transmit to the display AFF the ultrasound images acquired 11.

The device further comprises means for implementing a classifier CLASS such as described above. The classifier is configured to receive ultrasound images 11, associate a class 14 with ultrasound images in real time, and generate the quality indicator 13 in real time. The classifier is connected directly or indirectly to the display AFF to transmit the indicators generated to the display AFF.

According to an alternative, the classifier is implemented by remote electronic equipment, such as a remote server. In this case, the device comprises an interface to exchange data with the remote equipment in order to transmit data and retrieve the result of the processed, i.e. classified, data.

Finally, in the present invention, it is understood that when the classification is implemented wholly in part by remote equipment, the device of the invention can be interpreted as being the system comprising on the one hand the local device described in the present application and the remote means making it possible to implement the classification function.

The device further comprises at least one processor or a calculator CALC associated with a memory to execute at least a portion of the steps of the method according to the invention. For example, said processor may be configured to execute the steps of classifying 200, generating display of the quality indicator 300 and the position indicator 500 and/or automatically recording 300 of the sequence 20 of images. Said processor can be connected to the display AFF to transmit the position indicator 12 to the display AFF.

Device 1 may also comprise a plurality of processors, each associated with one or more memories, and configured to together execute such steps. In one embodiment, the processor(s) may be remote and connected to the display via a data network.

The device further comprises a memory for storing or recording sequences generated by the method according to the invention. In one embodiment, the device further comprises a transmitter EMM connected to said memory MEM to transmit said sequences recorded on said memory MEM to a data network.

The display AFF may comprise means to receive the different information 11, 13, 21, 12 received by the different means REC, CLASS, PROC of the device to generate the final image to display.

1. A guidance assistance device for acquiring ultrasound images, configured to receive in real time ultrasound images acquired by an ultrasound probe connected to said device, and comprising:
a first calculator, associated with a first memory, to implement a classifier configured to classify in real time the ultrasound images received and associate with each image a CLASS to generate a quality indicator according to said associated class;
a display to display, in real time, the last quality indicator generated;
a second calculator, associated with a second memory, and configured to perform the following steps:
determining a relative position of the ultrasound probe relative to a predefined probe target position; said relative position being determined from at least one of the received ultrasound images; and
generating and displaying on the display in real-time a position indicator according to the relative position determined.

2. The guidance assistance according to claim 1, wherein the position indicator displayed comprises a first visual element and a target mark; the position of said first element displayed on the display with respect to the target mark being a function of the relative position of the determined probe.

3. The guidance assistance according to claim 2, wherein the relative position determined comprises at least one relative orientation information of the probe with respect to a target orientation with respect to a predetermined axis of an orthonormal coordinate system and wherein the orientation of said first visual element with respect to the orientation of the target mark is a function of said relative orientation information.

4. The guidance assistance according to claim 2, wherein when the determined relative position reaches a predefined threshold value, the first visual element is confounded with the target mark.

5. The guidance assistance according to claim 2, wherein, when the acquired image is classified within the predefined image class, the first visual element is confounded with the target mark.

6. The guidance assistance according to claim 2, wherein the display is configured to display the received ultrasound images and wherein the target mark is arranged on the displayed ultrasound image in real time.

7. The guidance assistance according to claim 6, wherein the target mark comprises the contours of the displayed ultrasound image; wherein dimensions of the first visual element are similar to dimensions of the contours of the displayed ultrasound image.

8. The guidance assistance according to the claim 2, wherein the target mark comprises displaying a target and wherein the first visual element position with respect to a center of the target depends on the relative position determined.

9. The guidance assistance according to the claim 2, wherein the position indicator further comprises a 3-dimensional view of an organ; the target mark comprising a first target plane passing through said organ displayed and the first visual element comprising a second plane passing through said organ displayed.

10. The guidance assistance according to claim 1, configured to display the received ultrasound image in real time on the display and configured to display the position indicator superimposed on the received ultrasound image.

11. The guidance assistance according to claim 1, configured to display a representation of at least a portion of a body model on the display and configured to display the position indicator superimposed on said body model portion.

12. The guidance assistance according to claim 1, wherein said relative position comprises at least two spatial position coordinates representing the relative position of the probe head with respect to the predefined target position on the body surface of a subject.

13. The guidance assistance according to claim 1, wherein the second calculator is associated with a second memory for implementing a learning function configured to associate in real time with each image a relative position of the ultrasound probe with respect to a target position and to generate a position indicator as a function of said associated relative position.

14. The guidance assistance according to claim 13, wherein the learning function is trained with labeled training ultrasound images, each training ultrasound image being associated with a relative position of the ultrasound probe with respect to the predefined target position.

15. A guidance assistance method for acquiring computer-implemented ultrasound images comprising:
receiving ultrasound images from an ultrasound probe;
classifying in real time of the ultrasound images received, and associating each image with a class;
generating and displaying a quality indicator according to said class associated with the classified ultrasound image;
real time determination of a relative position of the ultrasonic probe relative to a predefined probe target position from each received ultrasound images; and
generating and displaying on the display in real time of a position indicator according to the relative position determined.

16. The guidance assistance method according to the claim 15, further comprising continuously displaying the ultrasound images received on a display.

17. A non-transitory computer program product comprising instructions which cause a device to perform the steps of the method according to claim 15.

18. A non-transitory computer-readable medium comprising instructions for performing the method according to claim 15.
